(19) United States
(12) Patent Application Publication
(10) Pub. No.: US 2022/0233694 A1
(43) Pub. Date: Jul. 28, 2022

(54) FUNCTIONALIZED FULLERENE GEL TUMOR TREATMENT

(71) Applicant: The Cleveland Clinic Foundation, Cleveland, OH (US)

(72) Inventor: Vijay Krishna, Beachwood, OH (US)

(21) Appl. No.: 17/612,115

(22) PCT Filed: May 29, 2020
(86) PCT No.: PCT/US2020/035063

§ 371 Date: Nov. 17, 2021

Related U.S. Application Data
(60) Provisional application No. 62/855,107, filed on May 31, 2019.

(51) Int. Cl. A61K 41/00; A61K 9/00; A61K 9/06; A61K 47/12; A61P 35/00

(52) U.S. Cl. CPC A61K 41/0028; A61K 9/0019; A61P 35/00; A61K 47/12; A61K 9/06

(57) ABSTRACT

Provided herein are compositions, systems, kits, and methods for administering a gel composition into a tumor of a subject and treating with laser light (e.g., for photoacoustic destruction of the tumor and tumor debris generation), where the gel comprises functionalized fullerenes (FFs) and a biocompatible polymer. In certain embodiments, 0.1-5% (e.g., about 1-2%) by weight of the gel is the functionalized fullerenes (e.g., polyhydroxy fullerenes). In other embodiments, the FFs have a generally symmetrical spherical structure.

The present application claims priority to U.S. Provisional application Ser. No. 62/855,107, filed May 31, 2019, which is herein incorporated by reference in its entirety.

### FIELD

Provided herein are compositions, systems, kits, and methods for administering a gel composition into a tumor of a subject and treating with laser light (e.g., for photoacoustic destruction of the tumor and tumor debris generation), where the gel comprises functionalized fullerenes (FFs) and a biocompatible polymer. In certain embodiments, 0.1-5% by weight of the gel is the functionalized fullerenes (e.g., polyhydroxy fullerenes). In other embodiments, the FFs have a generally symmetrical spherical structure.

### BACKGROUND

Every year, over 1.5 million new cases of cancers are diagnosed and over 600k cancer deaths are reported in the U.S [1]. Conventional treatment strategies of surgical resection, chemotherapy or radiation therapy do not activate anti-tumor immunity. Approaches to activate immune system against cancer has shifted the paradigm in cancer treatment. Current research efforts on cancer immunotherapy including a) cytokine therapy; b) adoptive cell transfer, including chimeric antigen receptor T (CAR-T); c) immune-checkpoint blockade; and d) vaccination have demonstrated exciting clinical responses [2-4]. Non-specific immune activation approaches, such as cytokine therapy or immune-checkpoint therapy have very low response rate (e.g., <25% for PD-LI positive and 5% for others) [5-7]. Further, non-specific activation of immune system often causes autoimmune diseases [8, 9]. Cytokine treatment, such as those using IFNα and IL2, CAR-T and immune-checkpoint blockade therapies can cause cytokine release syndrome or tumor lysis syndrome which lead to severe hypotension, renal dysfunction, seizures, arrhythmias and other adverse effects that are potentially lethal [10, 11]. Among above-mentioned cancer immunotherapy approaches, cancer vaccines provide several unique advantages [12-17]. Cancer vaccines with tumor-associated antigens or neoantigens induce antigen-specific immune response against tumors, rather than non-specific immunological responses triggered by other methods such as the checkpoint-blockade therapy response [12, 15, 18]. Further, cancer vaccines may offer a long-term immune-memory effect that could be helpful to prevent cancer recurrence [16]. Although, cancer vaccine created with specific neoantigens such as proteins or peptides may induce robust anti-tumor immune responses, the large heterogeneity of patients and tumors leads to their limited clinical applications [14, 15, 18].

Vaccination with whole tumor lysates (WTL) from surgically resected tumor is a conceptually attractive approach to mount robust immune response against all potential tumor antigens and, in principle, applicable to all types of solid tumors [19]. However, the sophistication and laboriousness of the treatment method, uncertainties in characteristics and dosages, as well as high-cost per patient has severely limited its clinical application [19-21]. The major limitations to immunotherapies are: 1) tumors have a strong immune-suppressive environment that antagonizes treatment strategies including vaccination; and 2) current treatments are systemic and lack approaches to localize to the tumor.

### SUMMARY

Provided herein are compositions, systems, kits, and methods for administering a gel composition into a tumor of a subject and treating with laser light (e.g., for photoacoustic destruction of the tumor and tumor debris generation), where the gel comprises functionalized fullerenes (FFs) and a biocompatible polymer. In certain embodiments, 0.1-5% by weight of the gel is the functionalized fullerenes (e.g., polyhydroxy fullerenes). In other embodiments, the FFs have a generally symmetrical spherical structure.

In some embodiments, provided herein are methods of treating a subject with a tumor comprising: a) administering a gel into an initial tumor of a subject such that a treated tumor is generated, wherein the gel comprises functionalized fullerenes (e.g., polyhydroxy fullerenes) and a biocompatible (e.g., biodegradable polymer); and b) subjecting the treated tumor to laser light. In certain embodiments, 0.1-5% (e.g., 0.5 . . . 1.0 . . . 1.5 . . . 2.0 . . . 2.5 . . . 3.5 . . . 4.0 . . . or 5.0%) or 0.1-10% by weight (e.g. 1% . . . 5% . . . 7.5% . . . 10%) of the gel is the functionalized fullerenes (e.g., polyhydroxy fullerenes). In some embodiments, the subject is treated with the laser light for 25 seconds to 35 minutes (e.g., 25 second 48 seconds . . . 2 minutes . . . 10 minutes . . . 20 minutes . . . 35 minutes), or 1-5 minutes. In certain embodiments, the volume of gel administered into the initial tumor is at least about 30% (e.g., 30% . . . 40% . . . or 48%) or at least about 50% of the initial tumor volume (e.g., 50% . . . 60% . . . 70% . . . or 95%). In certain embodiments, such as well a tumor is of a larger size, the tumor is treated a second, third, or fourth time (e.g., for 1-5 minutes each time).

In particular embodiments, provided herein are compositions comprising: functionalized fullerenes (e.g., polyhydroxy fullerenes) and a biocompatible (e.g., biodegradable) polymer, wherein the composition is in the form of a gel, and wherein 0.1-5% (e.g., 0.5 . . . 1.0 . . . 1.5 . . . 2.0 . . . 2.5 . . . 3.5 . . . 4.0 . . . or 5.0%) by weight of the composition is the functionalized fullerenes (e.g., polyhydroxy fullerenes).

In some embodiments, provided herein are kits or systems comprising: a) the compositions described herein; and b) a device that produces a laser.

In other embodiments, provided herein are methods of treating cancer in a subject with a tumor comprising: a) administering a composition into an initial tumor of a subject to generate a treated tumor, wherein the composition comprises nanoparticles coated with functionalized fullerenes (e.g., polyhydroxy fullerenes); and b) subjecting the treated tumor to laser light. In particular embodiments, the nanoparticles and the functionalized fullerenes (e.g., polyhydroxy fullerenes) are present in the composition at approximately equal weights (e.g., 40:60; 45:55; 50:50; 55:45; or 60:40).

In certain embodiments, the treatment causes the tumor to shrink in size (e.g., 30% . . . 50% . . . 95%). In other embodiments, the treatment causes the tumor to be completely eradicated. In other embodiments, the treatment prevents further tumors from forming. In some embodiments, the subjecting the treated tumor to laser light causes said tumor to shrink by at least 30 percent (e.g., at least 30 . . . 50 . . . 70 . . . 85 . . . 95 . . . 100%).

In some embodiments, 1-5% (e.g., 0.5 . . . 1.0 . . . 1.5 . . . 2.0 . . . 2.5 . . . 3.5 . . . 4.0 . . . or 5.0%) by weight of the gel is the biocompatible (e.g., biodegradable) polymer. In other embodiments, the biocompatible polymer comprises chitosan. In certain embodiments, the biocompatible polymer is selected from the group consisting of: chitosan, dextran, polyamidoamine (PAMAM), polylactic acid, polyglycolic acid, poly(lactic-co-glycolic) acid (PLGA), Eudragit and polycaprolactone (PCL). In further embodiments, 97.5-90.0% of the gel is water (e.g., 97.5 . . . 95.0 . . . 92.5 . . . or 90%).

In some embodiments, the fullerene cage of functionalized fullerenes (e.g., polyhydroxy fullerenes) have a generally symmetrical spherical structure. In other embodiments, the fullerene cage of FFs are selected from the following: C20, C24, C34, C36, C40, C44, C60, C72, C80, C82, C84, C96, C180, C240, C260, C320 and C540. In additional embodiments, the functionalized fullerenes have a cage structure without internal atoms (e.g., such that the symmetrical structure is preserved). In certain embodiments, the functionalized fullerenes are endohedral fullerenes. In some embodiments, the functionalized fullerenes are Gd@C60.

In some embodiments, the polyhydroxy fullerene is selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; and Gd₃N@C₈₀(OH)₁₃O₉Na₆. In other embodiments, the fullerene is selected from the group consisting of: a carboxyfullerene, an aminofullerene, a fullerene functionalized with amino acids, and a hexakis fullerene. In additional embodiments, 1-3% of said gel by weight is said functionalized fullerenes, and wherein 1.5-3.5% of said gel by weight is said biocompatible polymer. In further embodiments, the biocompatible polymer comprises chitosan or chitosan derivative. In some embodiments, the 0.5-1.5% of said gel by weight is said functionalized fullerenes, and wherein 1.0-3.0% of said gel by weight is said biocompatible polymer. In further embodiments, the biocompatible polymer comprises chitosan or chitosan derivative.

In some embodiments, provided herein are methods of treating a subject with a tumor comprising: a) administering a volume of gel into an initial tumor of a subject such that a treated tumor is generated, wherein said gel comprises functionalized fullerenes and a biocompatible polymer, and wherein said volume of gel administered is at least about 50% of said initial tumor volume (e.g., 50% . . . 55% . . . 60% . . . 65% . . . 75% . . . or 90%); and b) subjecting said treated tumor to laser light. In certain embodiments, the initial tumor is imaged (e.g., by MRI, CAT, etc.) to ascertain its volume prior to step a)).

In some embodiments, provided herein are compositions comprising: polyhydroxy fullerenes, a biocompatible polymer, and water, wherein the composition is in the form of a gel, wherein 1-4% by weight (e.g., 2-3% by weight) of said composition is said polyhydroxy fullerenes, wherein 1-4% by weight (e.g., 1-2% by weight) of the composition is the biocompatible polymer, and wherein the entire, or nearly entire, remaining percentage of the gel is water.

In certain embodiments, provided herein are methods of making a gel comprising: a) mixing a first composition with a second composition (e.g., vigorously) to generate a suspension, wherein said first composition comprises polyhydroxy fullerenes and water, and wherein said second composition comprises a biocompatible polymer and aqueous solvent, b) centrifuging said suspension to generate a supernatant liquid and a pellet in the form of a gel, and c) discarding said supernatant liquid to obtain said gel, wherein said gel comprises: i) 1-4% by weight of said polyhydroxy fullerenes, and ii) 1-4% by weight of said biocompatible polymer. In some embodiments, the aqueous solvent contains acid (e.g., acetic acid). In further embodiments, the polyhydroxy fullerenes are present in said first composition at about 10-20 mg/mL. In other embodiments, the biocompatible polymer is present in said aqueous solvent at about 1-10 mg/mL.

In some embodiments, the laser light has a wavelength of 250-2500 nm. In other embodiments, the wavelength is selected from the group consisting of: 350 nm, 532 nm, 600-650 nm, 700-950 nm, 700-990, 1000-1350 nm, 1600-1870, and 2100-2300 nm. In further embodiments, the laser light is blue, green, red, near-infrared, mid-infrared or far-infrared. For example, 405 nm, 532 nm, 600 nm, 650 nm, 740 nm, 785 nm, 808 nm, 810 nm, 980 nm, 1310 nm, 1550 nm and 10 μm. In certain embodiments has a wavelength of 785 nm or 808 nm.

In particular embodiments, the cancer type or tumor type is selected from the group consisting of: pancreatic cancer, breast cancer, myeloid cancers, lymphoid cancers (e.g., T-cell lymphoid cancers), small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

### DESCRIPTION OF THE FIGURES

The patent or application file contains at least one drawing executed in color. Copies of this patent or patent application publication with color drawings will be provided by the Office upon request and payment of the necessary fee.

FIG. 1: Hypothetical schematic showing how photoacoustic or mechanical destruction of tumor leads to cellular debris that provides multitude of neoantigens from heterogeneous tumor for priming of immune system.

FIG. 2: Exemplary comparison of certain embodiments of photoacoustic treatment with current state-of-the-art photothermal treatments.

FIG. 3: Changes in 4T1 tumor volume in female BALB/c mice. Treatment with Laser alone or Photoacoustic Gel (PAG) alone do not inhibit tumor growth. Photoacoustic treatment (PAG+Laser) leads to complete tumor regression with no recurrence for duration of study (120 days). (n=3)

FIG. 4: Photoacoustic treatment (I-III) and laser treatment (IV) of luciferase expressing 4T1 tumor in female BALB/c mice. A) Photoacoustic treatment completely destroys the tumor and no recurrence is observed for 21 days. B) After 21 days, a second luc-4T1 tumor was implanted on right side of the mouse. The tumor growth was inhibited and completely disappeared within 6 days after implantation. The second tumor did not receive any treatment. In contrast, control laser treatment was not able to inhibit growth of first or second tumors. (n=3)

FIG. 5: Immune response for control and photoacoustic treated mice as observed in blood withdrawn 1-week after treatment. The top row shows the percentages of CD11c gated CD80+ and CD86+ dendritic cells. The middle row shows the percentages of CD11b gated CD38+ and Egr2+ macrophages. The bottom row shows the percentages of CD8+ and CD4+ T cells.

FIG. 6: Magnetic resonance imaging with T2 contrast of tumor before and after treatment. Top Photoacoustic treatment completely destroys the tumor and induces an inflammatory response which shrinks the tumor. Bottom PAG alone induces little inflammation, however, not sufficient to inhibit tumor growth. The volume of tumor and inflammation is presented below each image in red and green, respectively.

FIG. 7 shows an exemplary multiple tumor model of a PhotoVaccine treatment (PVT) of luc-4T1 tumor in female BALB/c mouse. The mouse was implanted with two contralateral luc-4T1 tumors on Day −7. On Day 0, only left tumor received PVT by intratumoral administration of 30 μL PHF-chitosan gel followed by irradiation with 785 nm, 0.6 W laser for 10 minutes. The tumors were imaged at different timepoints. PVT rapidly destroys the treated tumor and no signal is observed. The untreated tumor grows up to Day 3 and then shrinks and disappears by Day 7 suggesting systemic immune response. Antigen recognition and priming of antigen-presenting cells (APCs) normally takes 3 days. The primed APCs activate cytotoxic T cells that can kill tumor cells. The peak in T cell response is usually observed 7-10 days after treatment.

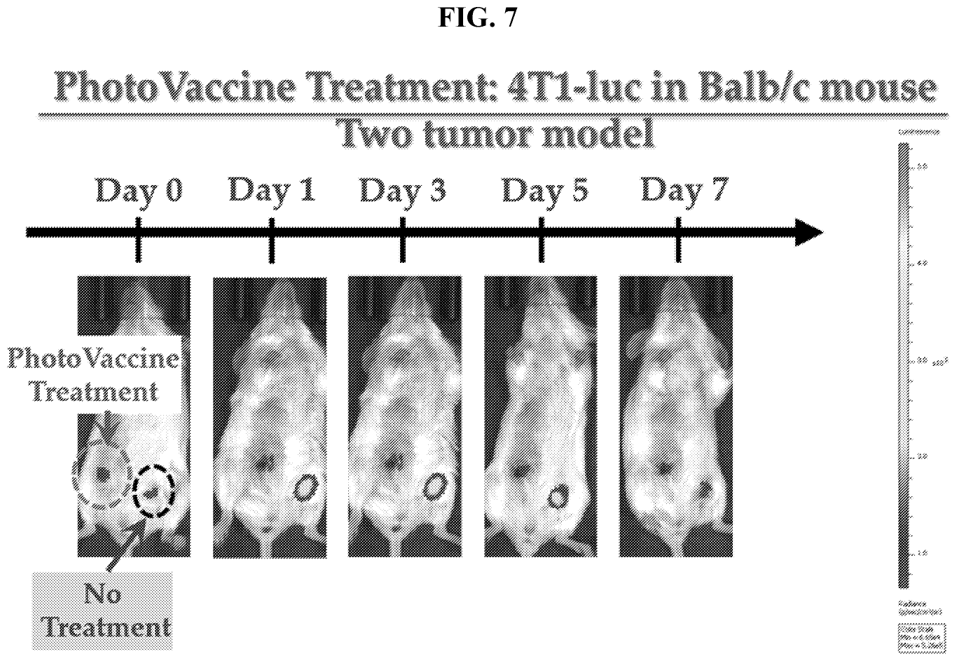

FIGS. 8A-C show clearance of fullerene gel after treatment. To visualize fate and clearance of fullerene gel after treatment, fluorescent fullerene gel was synthesized. Briefly, fluorescent dye Alexa Fluor 647 was first reacted with chitosan separately (C-AF647 conjugate). This conjugate was added to chitosan solution in 1% acetic acid. PHF was added and rapidly mixed to generate nanoparticles. The mixture was centrifuged and concentrated to obtain fluorescent fullerene gel. Optical and fluorescence photographs from IVIS for i) water; ii) fullerene gel without; and iii) fullerene gel with Alexa Fluor 647 dye is shown in FIG. 8A. This fluorescent fullerene gel was used in PVT as described earlier. Photographs of bioluminescent tumor before and 2 hours after PVT with fluorescent fullerene gel shows that addition of fluorescent dye does not interfere with treatment (FIG. 8B). Imaging after 2 hours, 2 days and 21 days of treatment shows that fluorescent fullerene gels are cleared from the tumor site (FIG. 8C).

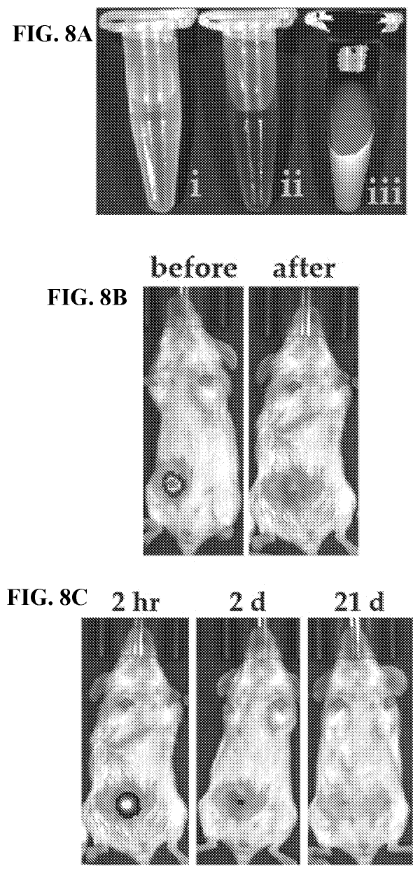

FIGS. 9A-B. In order to ascertain that fullerene gel does not affect proliferation of GL261 cells for co-implantation, in vitro experiments were carried out. In a centrifuge tube, equal volumes of GL261 cells (2.5×10\hat{ }6 cells/mL) and fullerene gel (1 mg/mL) were combined, vortexed and then injected (31 g insulin syringe) into ultra-low attachment, 96-well plate. To assess the cell viability, live-dead assay was performed at 24, 48 and 72 hr time points separately (n=3). The live-dead assay indicates that fullerene gel does not inhibit GL261 cell survival or proliferation (FIG. 9A). In fact, the fullerene gel act as a matrix for growth of GL261 cells. The mixture plated for 72 hours was exposed to near-infrared laser (785 nm; 500 mW) and imaged again to show that fullerene gel can kill GL261 cells (FIG. 9B).

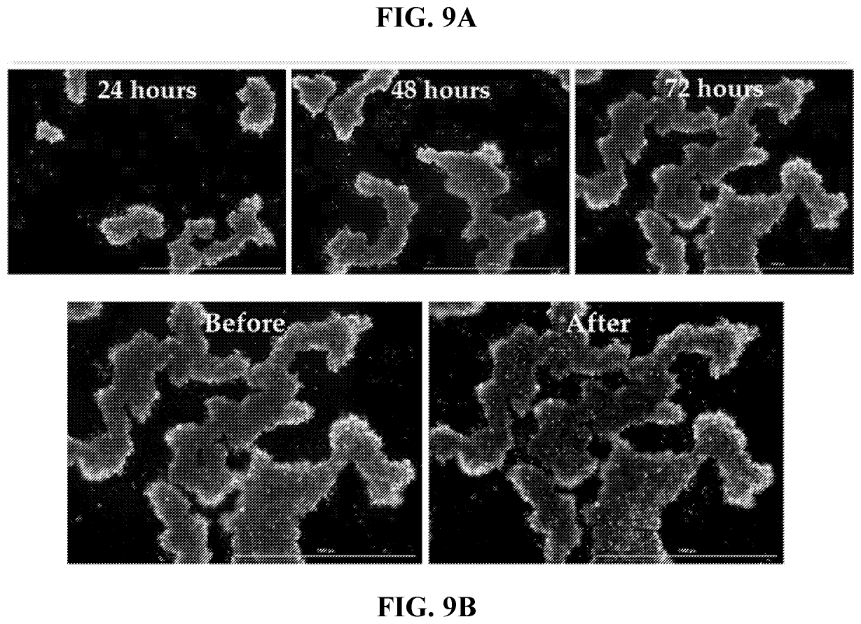

FIGS. 10A-B. In vivo experiments were carried out by intracranial injection of 10 μL of GL261 and fullerene gel mixture at a depth of 3 mm in frontal cortex. To prevent fullerene gel from diffusing out of location, the concentration was increased enough to form a viscous hydrogel that can be easily injected with 31 gauge syringe. Control mice received only GL261 cells. Two days after implantation, the tumors were imaged with MRI (0 d). Axial and coronal 2D T2-weighted turbo Rapid Acquisition with Refocused Echoes (RARE) images were acquired on 7T Bruker BioSpin 70/20, small animal MRI scanner. The fullerene gel appears bright in T2 images. Subsequently, the mice were exposed to near-infrared laser at 500 mW for 10 minutes. The mice were imaged again 1, 4 and 8 days after treatment. The MR images of mice brains were manually segmented and co-registered to the 0 d axial brain-masked image using FLIRT (FMRIB's Linear Image Registration Tool). The registered time-series for each mice is represented in FIGS. 10A-B. As evident from the MR images, no tumor is visible 4 days after photoacoustic treatment (PAT) and a necrotic region appears (region highlighted by yellow outline). However, tumor growth is visible in MR images of control mouse (region highlighted by white outline). It is also interesting to note that the fullerene gel, which appear bright in T2 images disappear within a week after intracranial implantation. FIG. 10A shows the timeline for tumor implantation, treatment and image acquisition. FIG. 10B, top row, shows Photoacoustic treatment destroys the tumor and a necrotic region is seen 4 and 8 days post-treatment. The GL261 cells+PANP region is highlighted with yellow outline. FIG. 10B, bottom row, shows laser alone does not inhibit growth of tumor. A mass of tumor is seen growing at 4 and 8 days post-treatment. The GL261 cells/tumor region is highlighted with orange outline.

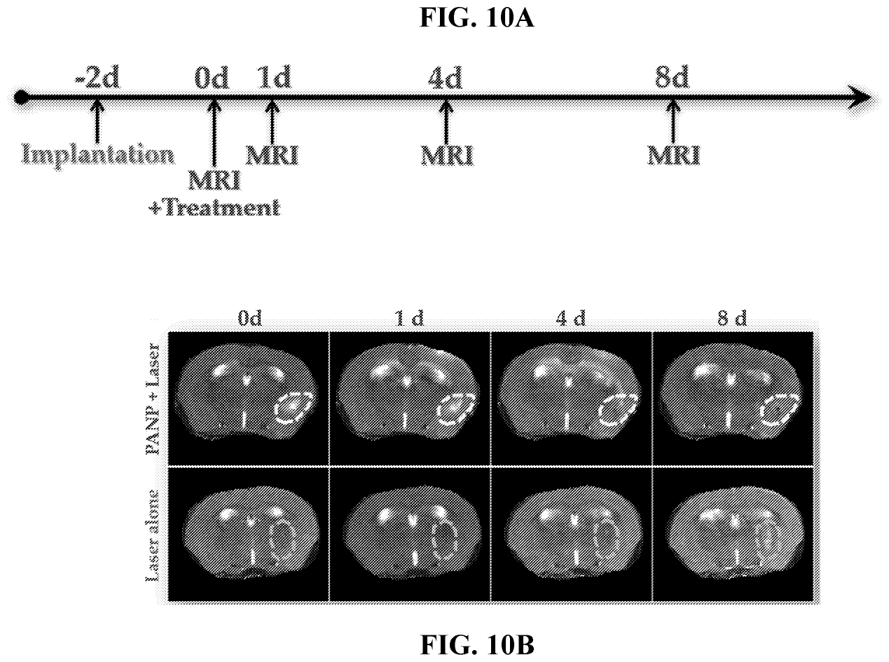

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. In addition, throughout the specification, the meaning of “a”, “an”, and “the” include plural references. The meaning of “in” includes “in” and “on.”

As used herein, the terms “subject” and “patient” refer to any animal, such as a mammal like a dog, cat, bird, livestock, and preferably a human.

As used herein, the term “administration” refers to the act of giving a drug, prodrug, or other agent (e.g., food product), or therapeutic treatment to a subject. Exemplary routes of administration to the human body can be through the mouth (oral), skin (transdermal, topical), nose (nasal), lungs (inhalant), oral mucosa (buccal), by injection (e.g., intravenously, subcutaneously, intratumorally, intraocular, intraperitoneally, etc.), and the like

As used herein, “fullerene” refers a general class of molecules that exists essentially in the shape of a three dimensional polyhedron containing from 20 to 1500 carbon atoms, and which comprises carbon atoms as the predominant element from which they are composed. The fullerenes include but are not limited to C-28, C-32, C-44, C-50, C-58, C-60, C-70, C-84, C-94, C-250 and C-540. In certain embodiments, the fullerenes are selected from: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₄O₁₄Na₁₇; C₆₀(OH)₁₃O₄Na₃; C₆₀(OH)₁₀; C₆₀(OH)₂₂₋₂₄; C₆₀(OH)₃₆; C₆₀(OH)₄₄; C₆₀O₁₃Na₁₄; Gd@C₈₂(OH)₁₅O₁₂Na₅; Gd₃N@C₈₀(OH)₁₃O₉Na₆; C₆₀(OH)₁₁O₈S₈Na₅; C₆₀(OH)₁₁(SH)₅O₈Na₅; C₆₀C₁₂N₄H₂₄; and C₆₀C₁₂N₆H₃₀O₁₂. According to this nomenclature, the fullerene which contains 60 carbon atoms is denoted C-60, the fullerene which contains 70 carbon atoms is denoted C-70, etc. Also included among the fullerenes are the substituted fullerenes. These are molecular fullerenes which have had one or more of the atoms which comprise the fullerene cage structure replaced by an atom other than carbon, such as nitrogen, boron or titanium, yet essentially retain the geometry of a polyhedron upon being so substituted. Also included among the fullerenes are endohedral fullerenes, in which atoms of elements other than carbon (e.g., iron, gadolinium and sulfur) reside inside the cage structure. Included in the term “fullerene” is a “functionalized fullerene” which refers to fullerene (Cₓ where x is 20 to 1500) with side groups attached to the outer surface of the cage via covalent bonds, ionic bonds, or Dewar coordination, or Kubas interactions, or any combination thereof. The side groups can be either inorganic, including, but not exclusive to, OH, Br, H₂, Gd, Ti, organic, including, but not exclusive to, C(COOH)₂, or any combination of organic and/or inorganic functional groups. The number of functional groups attached per cage of fullerene can vary from 1 to a majority of the number of carbons in the fullerene cage. Functionalized fullerenes have different physical and chemical properties based on the type and number of side groups. In certain embodiments, the fullerenes herein are compounds according to the formula C₂ₙ(OH)ₜ(SH)ᵤ(NH₂)ᵥ(COOH)_{w}(COOM)ₓO_{y}M_{z}, wherein M is an alkali metal, alkaline earth metal, transition metal, post-transition metal, lanthanide or actinide, n is a number ranging from 10 to 270; t, u, v, w, x, y and z can range from 0 to the total number of carbon atoms present in the cage. Examples of fullerenes are found in U.S. Pat. No. 9,950,977, which is herein incorporated by reference, in its entirety, particularly for the fullerene compounds disclosed therein. In certain embodiments, the fullerenes employed herein are polyhydroxy fullerenes (PHFs). PHF has hydroxyl and hemi-ketal groups appended to fullerene cage, and is a salt of alkaline metals and/or alkaline earth metals. For example, PHF can have formula of C₆₀(OH)₉O₇Na₆ or C₆₀(OH)₁₁O₂₀Na₁₀K₆ as determined by x-ray photoelectron spectroscopy.

### DETAILED DESCRIPTION

Provided herein are compositions, systems, kits, and methods for administering a gel composition into a tumor of a subject and treating with laser light (e.g., for photoacoustic destruction of the tumor and tumor debris generation), where the gel comprises functionalized fullerenes (FFs) and a biocompatible polymer. In certain embodiments, 0.1-5% by weight of the gel is the functionalized fullerenes (e.g., polyhydroxy fullerenes). In other embodiments, the FFs have a generally symmetrical spherical structure.

In some embodiments, the fullerenes comprise polyhydroxy fullerenes. In other embodiments, the fullerenes are compounds according to the formula C2n(OH)t(SH)u(NH2)v(COOH)w(COOM)xOyMz, wherein M is an alkali metal, alkaline earth metal, transition metal, post-transition metal, lanthanide or actinide, n is a number ranging from 10 to 270; and t, u, v, w, x, y and z can range from 0 to the total number of carbon atoms present in the cage. Exemplary polyhydroxy fullerenes are disclosed in U.S. Pat. Nos. 8,883,124, 9,475,028, 9,950,977, 9,084,989, and 9,731,013 (all five of which are herein incorporated by reference in their entireties, particularly for polyhydroxy fullerene formulas), and are used for generating photoacoustic gels and nanoparticles that generate nano-bursts for non-invasive mechanical destruction of tumor and in situ stimulation of immune system for cancer therapy.

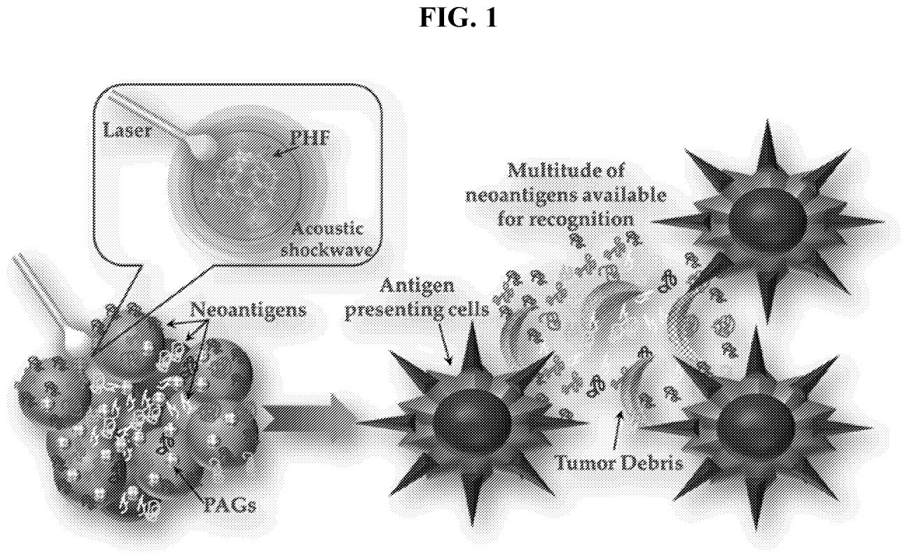

In certain embodiments, provides herein is a method for cancer immunotherapy using photoacoustic gels and nanoparticles for minimally-invasive, mechanical destruction of tumors to produce multitude of antigens that stimulate immune system irrespective of heterogeneity in tumor immunogenecity. Provided here are advantages such as: 1) a method for cancer immunotherapy; and 2) ability to provide personalized immunotherapy by in situ vaccination. Provided herein is the ability to use the unique optical properties of functionalized fullerenes (e.g., polyhydroxy fullerenes) [23] for engineering gels and nanoparticles that generate nano-bursts for minimally-invasive mechanical destruction of tumor and in situ stimulation of immune system for cancer therapy. While the present invention is not limited to any particular mechanism and an understanding of the mechanism is not necessary to practice the invention, in certain embodiments, the gels and nanoparticles provide the ability to: 1) generate photoacoustic damage without heating; 2) create minimally-invasive mechanical tumor destruction, which can provide multitude of neoantigens; and 3) stimulate the immune system against cancer in situ (FIG. 1).

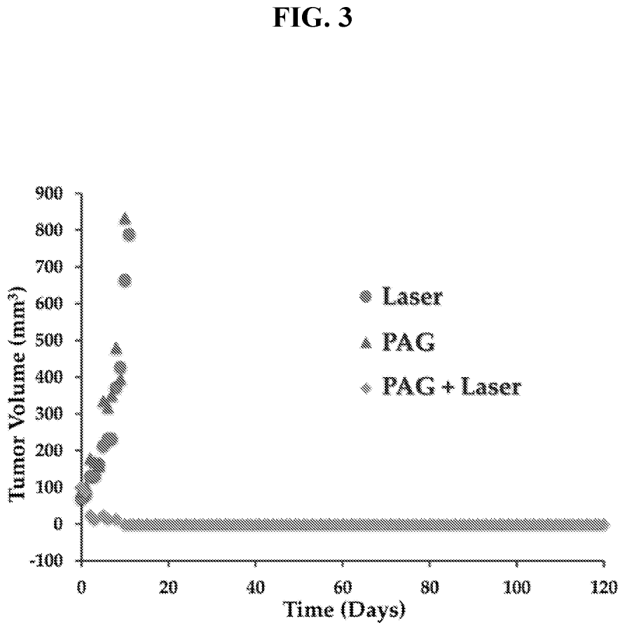

While not limited to any particular mechanism, it is believed that one of the important features is the ability to engineer light-to-sound, instead of light-to-heat, by controlling the structure of fullerenes (e.g., cage distortion and functional groups). We have engineered gels and nanoparticles with polyhydroxy fullerenes (PHFs) that produces acoustic shockwaves or nano bursts. In prior work, the differences in mechanical and thermal destruction was also demonstrated in vivo. Minimally-invasive treatment with Gd@C82 PHF resulted in photothermal destruction of tumor and scarring of skin (burn marks), with ˜40% tumor shrinkage in 24 hours [2, 3]. In contrast, in certain work conducted during development of embodiments herein, minimally-invasive photoacoustic treatment with C60 PHF shows no signs of skin damage and with only a blister and 100% tumor shrinkage after 24 hours (FIG. 3). Further, such work demonstrated that photoacoustic treatment prevents recurrence and inhibits growth of second tumor challenge.

In work conducted during the development of embodiments herein, gels with polyhydroxy fullerenes (PHF) produces acoustic shockwaves or nano bursts. In such work, we demonstrated minimally-invasive cancer treatment (FIG. 3) with rapid tumor destruction (˜50% shrinkage in 2 hours; 100% in 24 hours) in a murine model of breast cancer. Importantly, a single photoacoustic treatment with a near infrared laser of a primary tumor prevented growth of a second tumor implanted 21 days post-treatment. This response was observed without the use of costly chemo- or immune-adjuvants (e.g., in some embodiments, no other cancer agents are used to treat the subject, such as chemo or immune treatments), such as antibody-based checkpoint inhibitors. Immune response one-week after treatment suggest circulating dendritic cells and macrophages are altered.

Clinically used minimally-invasive treatment strategies for breast cancer include radiofrequency ablation, microwave ablation, high-intensity focused ultrasound and cryoablation that provide localized cancer treatment by changing the temperature of the tumor (hot or cold) to kill the breast cancer cells. Preclinical minimally invasive treatment strategies, such as photothermal treatment, utilize photothermal nanoparticles (metal, inorganic or polymer based) delivered to the tumor and exposed to deep-tissue penetrating near-infrared laser for heat generation and localized tumor destruction. The photothermal nanoparticles are delivered to the tumor by a) direct intratumoral injection, b) active targeting with antibody conjugated nanoparticles, or c) passive targeting with enhanced permeation and retention (EPR) effect. Clinical and pre-clinical minimally-invasive treatments result in coagulative necrosis of the tumor and exhibit an immune response, however, not sufficient enough to prevent recurrence and metastasis. Studies have associated release of danger associated molecular patterns and heat shock proteins such as HSP70, which act as antigen chaperones to APCs, with modest immune activation [24-26]. Minimally-invasive cancer treatment with photothermal nanoparticles in syngeneic tumor models have shown increase in dendritic cell maturation (CD11c gated CD80+CD86+) and cytotoxic T cells (CD3 gated CD4−CD8+), and decrease in regulatory T cells (CD3 gated CD4+FoxP3+) [27, 28]. Such thermal ablative procedures result in coagulative necrosis of the tumor, which also destroy antigens due to protein denaturation [29]. Immune activation observed is weak as coagulation process prevents release of intracellular antigens for recognition by APCs and the immune system is not primed for tumor heterogeneity [29]. To enhance the immunological profiles after photothermal treatment immune-adjuvants, such as anti-CTLA4 or glycated chitosan are necessary [27, 28, 30]. Photothermal treatment plus anti-CTLA4 treatment of syngeneic tumors increased serum level of TNFα and IFNγ, percentage of effector memory T cells (CD3 gated CD8+CD62L−CD44+), and reduced the percentage of central memory T cells (CD3 gated CD8+CD62L+CD44+).

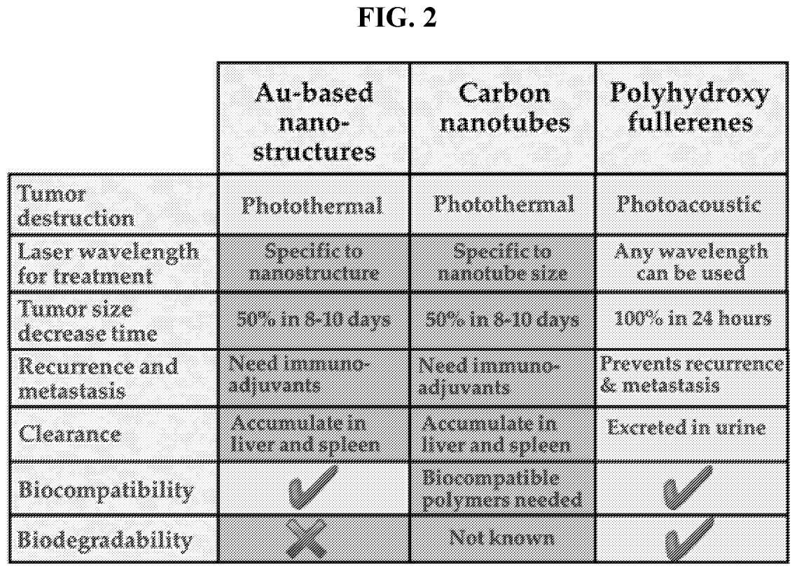

Exemplary advantages of photoacoustic treatment, based on work conducted herein, over current state-of-the-art photothermal treatments are threefold (FIG. 2). 1) Photoacoustic treatment results in rapid tumor destruction with complete or near complete inactivation of tumor within 24 hours post treatment. However, photothermal treatment results in 50% shrinkage of tumor in 8-10 days [31, 32]. 2) In certain embodiments, a single photoacoustic treatment is sufficient to prevent recurrence and growth of second tumor challenge. In contrast, photothermal treatments alone generally cannot prevent growth of second tumor challenge. Chemo- or immune-adjuvants are used along with photothermal treatments to prevent second tumor challenge [26-28, 30, 33, 34]. 3) Polyhydroxy fullerenes (PHF) used are non-toxic, easily cleared from the body and also known to extend lifespan in animals [35-37], which points to clinical translation. In contrast, photothermal nanoparticles, such as gold nanoshells, gold nanorods, carbon nanotubes and copper sulfide accumulate in liver and spleen with unknown fate and long-term effect [38-42].

In work conducted during development of embodiments herein, photoacoustic gels were produced by encapsulating C60 polyhydroxy fullerenes (PHF) in chitosan matrix. Briefly, 0.1 mL of PHF (10-20 mg/mL) was vigorously mixed with 0.9 mL chitosan (0.25 mg/mL or 2.5 mg/mL in 1% acetic acid). The resulting suspension was centrifuged at 300×g and supernatant was discarded. The pellet in the form of gel was used for in vivo experiments.

Other polymers may be used. For example, PHF encapsulation in Eudragit, dextran, PLGA and PCL polymers can follow a double emulsion method as follows. Prepare polymer matrix solution (e.g., 2-10 mg/mL Eudragit in methanol or PCL in methanol or PLGA in dichloromethane). Add 0.1 mL PHF (10-20 mg/mL) to 0.9 mL polymer matrix solution on ice and mix with pipette. Add the resulting emulsion to 9 mL of polyvinyl alcohol (0.1%; 13-23 kD) solution under vigorous stirring followed by sonication to achieve double emulsion. The double emulsion is stirred overnight to remove polymer solvents. The suspension is then washed three times with deionized water.

In order to determine the ability of photoacoustic treatment to mount anti-tumor immunity, an immune-competent and syngeneic model of breast cancer was chosen with 4T1 murine breast cancer cells orthotopically implanted in mammary fatpad of female BALB/c mice. Since 4T1 tumors are highly aggressive and exhibit rapid metastasis to lung, bone and brain, we conducted the photoacoustic treatment on tumors 4-6 mm in size. Such work demonstrated that photoacoustic treatment successfully inhibits tumor growth and no recurrence was observed for four months (duration of study) after the treatment (FIG. 3). In control experiments with gel alone or laser alone, tumor size increased as expected. While the present invention is not limited to any particular mechanism, and an understanding of the mechanism is not necessary to practice the invention, it is believed that the tumor debris created by photoacoustic treatment acts as a vaccine to prime immune system and mount anti-tumor response against future tumor challenge.

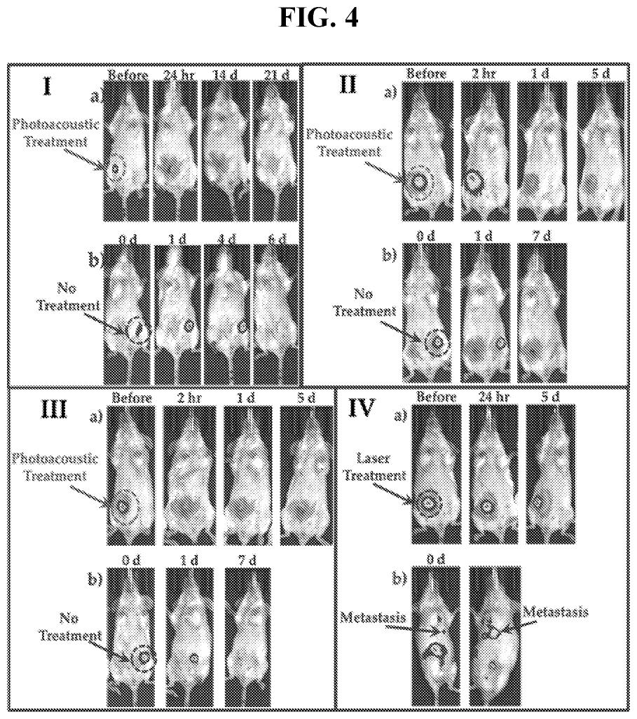

To assess the growth of secondary tumor, luciferase expressing 4T1 cells were utilized. Photoacoustic treatment of luc-4T1 tumors results in complete tumor destruction within 24 hours of the treatment and no recurrence was observed for the next 21 days (FIG. 4a). To evaluate the generation of anti-4T1 immune responses, luc-4T1 cells were implanted orthotopically on right side of the same mouse 21 days after the treatment. As seen from FIG. 4b, the newly implanted tumor cells completely disappeared within 6 days of implantation. Most importantly, no recurrence was observed for four months (duration of study), strongly suggesting the existence of robust anti-4T1 immunity capable of regressing the second tumor challenge. Laser alone or PAG alone did not inhibit growth of first or second tumor.

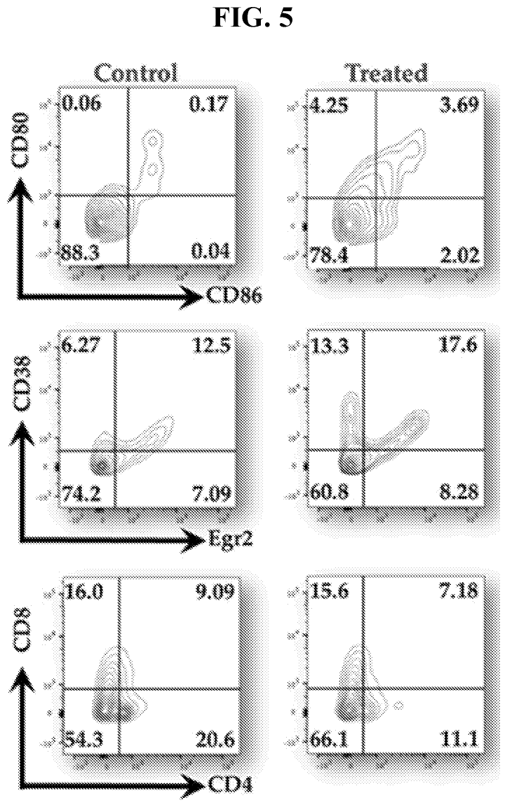

Work conducted during development of embodiments herein demonstrated that photoacoustic treatment elicits anti-tumor immune responses that effectively prevents the growth of secondarily challenged tumor cells. Combinatorial minimally-invasive cancer treatment with photothermal plus immune-adjuvants in syngeneic tumor models have shown increase in dendritic cell maturation (CD11c gated CD80+CD86+), cytotoxic T cells (CD3 gated CD4−CD8+), effector memory T cells (CD3 gated CD8+CD62L−CD44+), and decrease in regulatory T cells (CD3 gated CD4+FoxP3+) [27, 28]. Unlike photothermal treatment, photoacoustic treatment herein does not, in certain embodiments, require immunoadjuvants to prevent the growth of second tumor. This observation suggests that photoacoustic treatment results in robust immune response. In work conducted during development of embodiment herein, immunological studies established the protocol for harvesting blood, lymph node and spleen and developed baselines for characterizing DCs, macrophages and T cells. In one set of experiments we examined blood samples drawn from saphenous vein for control and photoacoustic treated mice 1-week after the treatment (FIG. 5). The proportions of mature (CD11c gated CD80+CD86+) DCs were higher in the blood of treated vs control mice. The macrophages were gated based on CD11b and F4/80 expression and further analyzed for CD38 and Egr2 expression to determine the proportions of M1 (CD38+Egr2−) and M2 (CD38−Egr2+) polarized macrophages [43]. The proportions of M1 (CD38+Egr2−) polarized macrophages was higher in the blood of treated mice than control. The CD4 T cells were higher in control mice than treated, however, the CD8 T cell proportions were similar between the control and the treated mice and this could be because of the timepoint and tissue chosen.

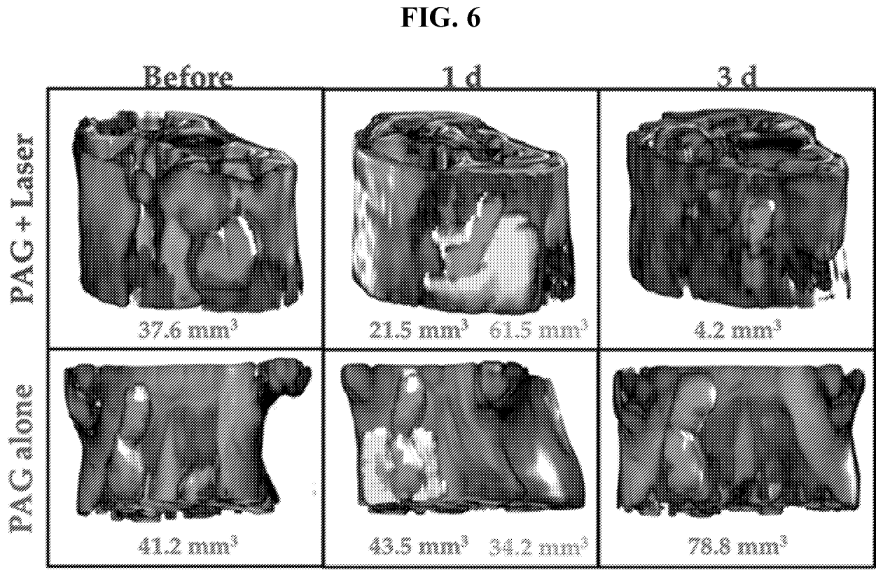

In work conducted during development of embodiments herein, we have demonstrated the feasibility of photoacoustic treatment in an immune-competent and syngeneic model of brain cancer with CT-2a murine glioblastoma cells implanted heterotopically on the flank of C57BL6/j mice. Photoacoustic gel (PAGs) were injected directly into the tumor (6-8 mm) followed by irradiation with NIR laser (300 J/cm2). Magnetic resonance imaging with T2 contrast was acquired before the treatment and 1 day and 3 days post treatment. The MR image analysis demonstrate that photoacoustic treatment successfully destroys the tumor (FIG. 6) with complete tumor disappearance within a week. In contrast, control experiments with gel PAGs alone do not inhibit tumor growth. Importantly, MRI shows the presence of fluid surrounding the tumor after photoacoustic treatment that suggests immune response to the treatment.

In certain embodiments, rather than a gel, the functionalized fullerenes (e.g., PHFs) are coated onto nanoparticles. Functionalized fullerenes can be coated on, for example, on inorganic nanoparticles (e.g., silica) and metallic nanoparticles (e.g., gold). In some embodiments, the nanoparticles are silica. Silica nanoparticles (normal or mesoporous) may be suspended in ethanol (5 mg/mL) and 800 microliter of APTS added dropwise and allowed to react. The resultant positively charged aminated silica nanoparticles are washed three times with water. Subsequently, functionalized fullerenes (e.g., 10-20 mg/mL) is added to aminated silica nanoparticles (1:1 wt ratio) and washed with water.

### REFERENCES

1. American Cancer Society. Cancer Facts & Figures 2018. 2018.
2. R. S. Riley, et al., Delivery technologies for cancer immunotherapy. Nat Rev Drug Discov, 2019.
3. P. Sharma and J. P. Allison, The future of immune checkpoint therapy. Science, 2015. 348(6230): p. 56-61.
4. C. G. Drake, E. J. Lipson, and J. R. Brahmer, Breathing new life into immunotherapy: review of melanoma, lung and kidney cancer. Nat Rev Clin Oncol, 2014. 11(1): p. 24-37.
5. H. Shi, et al., The status, limitation and improvement of adoptive cellular immunotherapy in advanced urologic malignancies. Chin J Cancer Res, 2015. 27(2): p. 128-37.
6. L. Wein, et al., Checkpoint blockade in the treatment of breast cancer: current status and future directions. Br J Cancer, 2018. 119(1): p. 4-11.
7. P. Schmid, et al., Atezolizumab and Nab-Paclitaxel in Advanced Triple-Negative Breast Cancer. N Engl J Med, 2018.
8. L. Calabrese and X. Mariette, The evolving role of the rheumatologist in the management of immune-related adverse events (irAEs) caused by cancer immunotherapy. Ann Rheum Dis, 2018. 77(2): p. 162-164.
9. M. Tocut, R. Brenner, and G. Zandman-Goddard, Autoimmune phenomena and disease in cancer patients treated with immune checkpoint inhibitors. Autoimmun Rev, 2018. 17(6): p. 610-616.
10. L. J. Burns, et al., IL-2-based immunotherapy after autologous transplantation for lymphoma and breast cancer induces immune activation and cytokine release: a phase I/II trial. Bone Marrow Transplant, 2003. 32(2): p. 177-86.
11. C. H. June, J. T. Warshauer, and J. A. Bluestone, Is autoimmunity the Achilles' heel of cancer immunotherapy? Nat Med, 2017. 23(5): p. 540-547.
12. S. A. Rosenberg, Decade in review-cancer immunotherapy: entering the mainstream of cancer treatment. Nat Rev Clin Oncol, 2014. 11(11): p. 630-2.
13. A. Ardiani, et al., Vaccine-mediated immunotherapy directed against a transcription factor driving the metastatic process. Cancer Res, 2014. 74(7): p. 1945-57.
14. J. A. Brinkman, et al., Peptide-based vaccines for cancer immunotherapy. Expert Opin Biol Ther, 2004. 4(2): p. 181-98.
15. Derin B. Keskin, et al., Neoantigen vaccine generates intratumoral T cell responses in phase Ib glioblastoma trial. Nature, 2018.
16. P. L. Lollini, et al., Vaccines for tumour prevention. Nat Rev Cancer, 2006. 6(3): p. 204-16.
17. P. A. Ott, et al., An immunogenic personal neoantigen vaccine for patients with melanoma. Nature, 2017. 547(7662): p. 217-221.
18. Norbert Hilf, et al., Actively personalized vaccination trial for newly diagnosed glioblastoma. Nature, 2018.
19. C. L. Chiang, G. Coukos, and L. E. Kandalaft, Whole Tumor Antigen Vaccines: Where Are We? Vaccines (Basel), 2015. 3(2): p. 344-72.
20. N. Zacharakis, et al., Immune recognition of somatic mutations leading to complete durable regression in metastatic breast cancer. Nat Med, 2018. 24(6): p. 724-730.
21. L. Cicchelero, H. de Rooster, and N. N. Sanders, Various ways to improve whole cancer cell vaccines. Expert Review of Vaccines, 2014. 13(6): p. 721-735.
22. R. H. Vonderheide, S. M. Domchek, and A. S. Clark, Immunotherapy for Breast Cancer: What Are We Missing? Clin Cancer Res, 2017. 23(11): p. 2640-2646.
23. V. Krishna, et al., Polyhydroxy fullerenes for non-invasive cancer imaging and therapy. Small, 2010. 6(20): p. 2236-2241.
24. L. S. Teng, et al., Radiofrequency ablation, heat shock protein 70 and potential anti-tumor immunity in hepatic and pancreatic cancers: a minireview. Hepatobiliary Pancreat Dis Int, 2010. 9(4): p. 361-5.
25. R. Rai, et al., Study of apoptosis and heat shock protein (HSP) expression in hepatocytes following radiofrequency ablation (RFA). J Surg Res, 2005. 129(1): p. 147-51.
26. Y. Li, et al., Nanotechnology-based photoimmunological therapies for cancer. Cancer Lett, 2019. 442: p. 429-438.
27. Q. Chen, et al., Photothermal therapy with immune-adjuvant nanoparticles together with checkpoint blockade for effective cancer immunotherapy. Nat Commun, 2016. 7: p. 13193.
28. L. Guo, et al., Combinatorial photothermal and immuno cancer therapy using chitosan-coated hollow copper sulfide nanoparticles. ACS Nano, 2014. 8(6): p. 5670-81.
29. K. F. Chu and D. E. Dupuy, Thermal ablation of tumours: biological mechanisms and advances in therapy. Nat Rev Cancer, 2014. 14(3): p. 199-208.
30. F. Zhou, et al., Local Phototherapy Synergizes with Immunoadjuvant for Treatment of Pancreatic Cancer through Induced Immunogenic Tumor Vaccine. Clin Cancer Res, 2018. 24(21): p. 5335-5346.
31. D. P. O'Neal, et al., Photo-thermal tumor ablation in mice using near infrared-absorbing nanoparticles. Cancer Lett, 2004. 209(2): p. 171-6.
32. A. M. Gobin, et al., Near-infrared resonant nanoshells for combined optical imaging and photothermal cancer therapy. Nano Lett, 2007. 7(7): p. 1929-34.
33. A. S. Bear, et al., Elimination of metastatic melanoma using gold nanoshell-enabled photothermal therapy and adoptive T cell transfer. PLoS One, 2013. 8(7): p. e69073.
34. Y. Liu, et al., Synergistic Immuno Photothermal Nanotherapy (SYMPHONY) for the Treatment of Unresectable and Metastatic Cancers. Sci Rep, 2017. 7(1): p. 8606.
35. Z. Ji, et al., Biodistribution and tumor uptake of C60(OH)x in mice. Journal of Nanoparticle Research, 2006. 8: p. 53-63.
36. J. Gao, et al., Polyhydroxy Fullerenes (Fullerols or Fullerenols): Beneficial Effects on Growth and Lifespan in Diverse Biological Models. Plos One, 2011. 6(5).
37. J. Wang, et al., Antioxidative function and biodistribution of [Gd@C82(OH)22]n nanoparticles in tumor-bearing mice. Biochem Pharmacol, 2006. 71(6): p. 872-81.
38. G. von Maltzahn, et al., Computationally guided photothermal tumor therapy using long-circulating gold nanorod antennas. Cancer Res, 2009. 69(9): p. 3892-900.
39. H. S. Choi, et al., Renal clearance of quantum dots. Nat Biotechnol, 2007. 25(10): p. 1165-70.
40. W. D. James, et al., Application of INAA to the build-up and clearance of gold nanoshells in clinical studies in mice. Journal of Radioanalytical and Nuclear Chemistry, 2007. 271(2): p. 455-459.
41. K. Kostarelos, A. Bianco, and M. Prato, Promises, facts and challenges for carbon nanotubes in imaging and therapeutics. Nature Nanotechnology, 2009. 4(10): p. 627-633.
42. W. Gao, et al., Copper sulfide nanoparticles as a photothermal switch for TRPV1 signaling to attenuate atherosclerosis. Nat Commun, 2018. 9(1): p. 231.
43. K. A. Jablonski, et al., Novel Markers to Delineate Murine M1 and M2 Macrophages. PLoS One, 2015. 10(12): p. e0145342.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of treating a subject with a tumor comprising:
a) administering a gel into an initial tumor of a subject such that a treated tumor is generated,
wherein said gel comprises functionalized fullerenes and a biocompatible polymer; and
b) subjecting said treated tumor to laser light.

2. The method of claim 1, wherein 0.1-5% by weight of said gel is said functionalized fullerenes.

3. The method of claim 1, wherein 1-5% by weight of said gel is said biocompatible polymer.

4. The method of claim 1, wherein said functionalized fullerenes are polyhydroxy fullerenes.

5. The method of claim 1, wherein said biocompatible polymer is selected from the group consisting of: chitosan, dextran, PAMAM, PLGA, Eudragit and PCL.

6. The method of claim 1, wherein 97.5-90.0% of said gel is water.

7. The method of claim 1, wherein said functionalized fullerenes have a generally symmetrical spherical structure.

8. The method of claim 7, wherein said functionalized fullerenes are selected from the following: C20, C24, C34, C36, C40, C44, C60, C72, C80, C82, C84, C96, C180, C240, C260, C320 and C540.

9. The method of claim 1, wherein said the volume said gel administered into said initial tumor is at least about 50% of said initial tumor volume.

10. The method of claim 1, wherein said laser light has a wavelength of 250-2500 nm, 785 nm, or 808 nm.

11. The method of claim 11, wherein said subjecting said treated tumor to laser light causes said tumor to shrink by at least 30 percent.

12. The method of claim 4, wherein said polyhydroxy fullerene is selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; and Gd₃N@C₈₀(OH)₁₃O₉Na₆.

13. The method of claim 1, wherein said fullerene is selected from the group consisting of: a carboxyfullerene, an aminofullerene, a fullerene functionalized with amino acids, and a hexakis fullerene.

14. The method of claim 1, wherein 1-3% of said gel by weight is said functionalized fullerenes, and wherein 1.5-3.5% of said gel by weight is said biocompatible polymer.

15. The method of claim 14, wherein said biocompatible polymer comprises chitosan and said tumor is a breast cancer tumor.

16. The method of claim 1, wherein 0.5-1.5% of said gel by weight is said functionalized fullerenes, and wherein 1.0-3.0% of said gel by weight is said biocompatible polymer.

17. The method of claim 14, wherein said biocompatible polymer comprises chitosan and said tumor is a glioblastoma tumor.

18. A composition comprising: functionalized fullerenes and a biocompatible polymer, wherein said composition is in the form of a gel, and wherein 0.1-5% by weight of said composition is said functionalized fullerenes.

19. The composition of claim 18, wherein 1-5% by weight of said composition is said biocompatible polymer.

20. The composition of claim 18, wherein said functionalized fullerenes are polyhydroxy fullerenes.

21. The composition of claim 18, wherein said biocompatible polymer is selected from the group consisting of: chitosan, PLGA, Eudragit and PCL.

22. The composition of claim 18, wherein 97.5-90.0% of said composition is water.

23. The composition of claim 18, wherein said functionalized fullerenes have a generally symmetrical spherical structure.

24. The composition of claim 23, wherein the functionalized fullerenes are selected from the following: C20, C24, C34, C36, C40, C44, C60, C72, C80, C82, C84, C96, C180, C240, C260, C320 and C540.

30. The composition of claim 24, wherein said functionalized fullerenes have a cage structure without internal atoms or are endohedral fullerenes.

31. The composition 20, wherein said polyhydroxy fullerene is selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; and Gd₃N@C₈₀(OH)₁₃O₉Na₆.

32. The composition of claim 18, wherein said fullerene is selected from the group consisting of: a carboxyfullerene, an aminofullerene, a fullerene functionalized with amino acids, and a hexakis fullerene.

33. The composition of claim 18, wherein 1-3% of said gel by weight is said functionalized fullerenes, and wherein 1.5-3.5% of said gel by weight is said biocompatible polymer.

34. The composition of claim 33, wherein said biocompatible polymer comprises chitosan.

35. The composition of claim 18, wherein 0.5-1.5% of said gel by weight is said functionalized fullerenes, and wherein 1.0-3.0% of said gel by weight is said biocompatible polymer.

36. The composition of claim 35, wherein said biocompatible polymer comprises chitosan.

37. A kit or system comprising:
a) said composition of claim 18; and
b) a device that produces a laser.

38. A method of treating cancer in a subject with a tumor comprising:
a) administering a composition into an initial tumor of a subject to generate a treated tumor,
wherein said composition comprises nanoparticles coated with functionalized fullerenes; and
b) subjecting said treated tumor to laser light.

39. The method of claim 38, wherein said nanoparticles and said functionalized fullerenes are present in said composition at approximately equal weights.

40. The method of claim 38, wherein said functionalized fullerenes are polyhydroxy fullerenes.

41. The method of claim 38, wherein said subjecting said treated tumor to laser light causes said tumor to shrink by at least 30 percent.

42. A method of treating a subject with a tumor comprising:
a) administering a volume of gel into an initial tumor of a subject such that a treated tumor is generated,
wherein said gel comprises functionalized fullerenes and a biocompatible polymer, and
wherein said volume of gel administered is at least about 50% of said initial tumor volume; and
b) subjecting said treated tumor to laser light.

43. The method of claim 42, wherein 1-3% of said gel by weight is said functionalized fullerenes, and wherein 1.5-3.5% of said gel by weight is said biocompatible polymer.

44. The method of claim 43, wherein said biocompatible polymer comprises chitosan and said tumor is a breast cancer tumor.

45. The method of claim 42, wherein 0.5-1.5% of said gel by weight is said functionalized fullerenes, and wherein 1.0-3.0% of said gel by weight is said biocompatible polymer.

46. The method of claim 45, wherein said biocompatible polymer comprises chitosan and said tumor is a glioblastoma tumor.

47. The method of claims 43-46, wherein the entire, or nearly entire, remaining percentage of said gel is water.

48. The method of claim 42, wherein said functionalized fullerenes are selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; and Gd₃N@C₈₀(OH)₁₃O₉Na₆.

49. The method of claim 42, wherein said subjecting said treated tumor to laser light is conducted for 1-5 minutes, and/or the laser light has a frequency of 785 nm or 808 nm.

50. A composition comprising: polyhydroxy fullerenes, a biocompatible polymer, and water, wherein said composition is in the form of a gel,
wherein 1-4% by weight of said composition is said polyhydroxy fullerenes,
wherein 1-4% by weight of said composition is said biocompatible polymer, and
wherein the entire, or nearly entire, remaining percentage of said gel is water.

51. The composition of claim 50, wherein said biocompatible polymer comprises chitosan.

52. The composition of claim 50, wherein said polyhydroxy fullerenes are selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; Gd₃N@C₈₀(OH)₁₃O₉Na₆, and mixtures thereof.

53. The composition of claim 50, wherein about 2-3% of said gel is said biocompatible polymer.

54. The composition of claim 50, wherein about 1-2% of said gel is said polyhydroxy fullerenes.

55. A method of making a gel comprising:
a) mixing a first composition with a second composition to generate a suspension,
wherein said first composition comprises polyhydroxy fullerenes and water, and
wherein said second composition comprises a biocompatible polymer and an aqueous solvent,
b) centrifuging said suspension to generate a supernatant liquid and a pellet in the form of a gel, and
c) discarding said supernatant liquid to obtain said gel, wherein said gel comprises: i) 1-4% by weight of said polyhydroxy fullerenes, and ii) 1-4% by weight of said biocompatible polymer.

56. The method of claim 55, wherein said aqueous solvent comprises acetic acid.

57. The method of claim 55, wherein said polyhydroxy fullerenes are present in said first composition at about 10-20 mg/mL.

58. The method of claim 55, wherein said biocompatible polymer is present in said aqueous solvent at about 1-10 mg/mL.

59. The method of claim 55, wherein said biocompatible polymer comprises chitosan.

60. The method of claim 55, wherein said polyhydroxy fullerenes are selected from the group consisting of: C₆₀(OH)₉O₇Na₆; C₆₀(OH)₁₁O₈Na₅; C₆₀(OH)₁₁O₁₂Na₈; C₆₀(OH)₁₁O₂₀Na₁₀K₆; C₆₀(OH)₆O₄Na₄; C₆₀(OH)₂₀O₈Na₄; C₆₀(OH)₁₀O₁₃Na₆; C₆₀(OH)₁₃O₄Na₃; C₆₀(OH)₂₂₋₂₄; C₆₀(OH)₃₆; Gd@C₈₂(OH)₁₅O₁₂Na₅; Gd₃N@C₈₀(OH)₁₃O₉Na₆, and mixtures thereof.
